(19) United States
(12) Patent Application Publication
(10) Pub. No.: US 2026/0263244 A1
(43) Pub. Date: Sep. 10, 2026

(54) SYSTEMS FOR REAL-TIME PREDICTION OF TRANSITIONS TO RIGID AND COMPLIANT SURFACES USING MYOELECTRIC AND KINEMATIC DATA

(71) Applicant: UNIVERSITY OF DELAWARE, Newark, DE (US)

(72) Inventors: Panagiotis Artemiadis, Hockessin, DE (US); Charikleia Angelidou, Newark, DE (US)

(73) Assignee: University of Delaware, Newark, DE (US)

(21) Appl. No.: 19/167,505

(22) PCT Filed: Mar. 22, 2024
(86) PCT No.: PCT/US2024/021108

§ 371 Date: Sep. 22, 2025

Related U.S. Application Data
(60) Provisional application No. 63/453,928, filed on Mar. 22, 2023.

(51) Int. Cl. A61F 2/72; A61B 5/00; A61B 5/11; A61B 5/397; A61F 2/66; A61F 2/76

(52) U.S. Cl. CPC A61F 2/72; A61B 5/112; A61B 5/397; A61B 5/7267; A61F 2/6607; A61F 2002/7685

(57) ABSTRACT

An electronic control system of a lower limb prosthesis includes a control device, a plurality of sensors, and a controller. The plurality of sensors captures kinematic data and electromyographic (EMG) recordings during at least one gait cycle. The gait cycle has at least one of: a rigid (R) terrain scenario corresponding to movement between a rigid surface and another rigid surface, and a transition (T) terrain scenario corresponding to movement between the rigid surface and a compliant surface. The controller processes the kinematic data and EMG recordings, then performs a classification thereof as corresponding to the R terrain or T terrain scenario, and predicts a subject's intent to move in accordance with the R terrain or T terrain scenario. The controller is configured to dynamically modify, in real-time, one or more control settings of the control device in response to the classification of the kinematic data and EMG recordings.

### CROSS-REFERENCE TO RELATED APPLICATIONS

This application claims priority to U.S. Provisional Application No. 63/453,928, filed Mar. 22, 2023, the content of which is incorporated herein by reference in its entirety for all purposes.

### REFERENCE TO U.S. GOVERNMENT SUPPORT

This invention was made with government support under Grant Nos. 2020009, 2015786, 2025797, and 2018905 from the National Science Foundation. The United States has certain rights in the invention.

### FIELD OF THE INVENTION

Aspects of the invention relate generally to lower limb prosthetic devices. More particularly, aspects of the invention relate to an electronic control system for a powered lower limb of a subject.

### BACKGROUND OF THE INVENTION

Locomotion is a crucial component involved in almost all daily activities. Thus, loss of either one or both lower limbs entails a series of long-term physical and psychological challenges for individuals with gait impairment, with major issues pertaining to balance, falling and the fear of falling. In particular, walking surfaces of varying compliance are encountered frequently in everyday life, making locomotion for people with lower-limb amputation strenuous. Current methodologies for powered ankle prostheses have replicated conditions for walking and running, but environmental information regarding the type of walking terrain and ground stiffness has been generally overlooked. Human control of locomotion requires information about the walking environment, which is usually obtained via proprioceptive mechanisms preceding movement execution. Able-bodied humans can anticipate the future state of the walking terrain and integrate this information when preparing motor responses, e.g., predict and adjust their next step to the walking terrain prior to transitioning from a more rigid to a more compliant surface.

Prior works relating to anticipation and user intent prediction primarily focus on locomotion-mode identification using electromyography (EMG) signals, mechanical data, or a combination thereof. However, the close resemblance of the muscle activation and body mechanics between rigid and compliant surface transitions, as well as the natural variability in a subject's walking pattern increases the difficulty in determining anticipation and user intent prediction preceding transitions between more rigid to more compliant surfaces. In addition, current control methodologies for powered ankle prostheses have successfully replicated conditions for walking on rigid surfaces. However, agility and walking stability on non-flat and compliant surfaces remain a significant challenge for individuals with gait impairment. Tuning the control parameters of a powered prostheses to adapt to new surfaces permits prosthesis/orthosis wearers to ambulate smoothly and adapt to changing environments. This increases the robustness and safety of prostheses as well as improve quality of life of individuals living with an amputation, e.g. a lower limb amputation.

Therefore, there remains a desire for improvements in gait performance and stability provided by lower limb prosthetic devices, including improvements in control of parameters related to transition between surfaces of different compliances.

### SUMMARY OF THE INVENTION

According to one aspect of the invention, an electronic control system is disclosed. The electronic control system is configured for a powered lower limb of a subject. The electronic control system includes a control device, a plurality of sensors, and a controller. The control device is configured for one or more joints of the lower limb. The plurality of sensors are attached to the subject and configured to capture kinematic data and electromyographic (EMG) recordings during at least one gait cycle performed by the subject. The at least one gait cycle comprises at least one of: a rigid (R) terrain scenario corresponding to movement or preparation for movement of the subject between a rigid surface and another rigid surface, and a transition (T) terrain scenario corresponding to movement or preparation for movement of the subject between the rigid surface and a non-rigid or compliant surface. The controller is coupled to the control device. Additionally, the controller is configured to control one or more control settings of the control device during the at least one gait cycle in accordance with instructions stored in a digital memory. The controller is configured to process the kinematic data related to motion of the subject. The kinematic data includes data related to the motion of one or more of a hip joint, a knee joint, and an ankle joint of the subject during the at least one gait cycle performed by the subject. The controller is also configured to process the surface EMG recordings of one or more of the subject's muscles during the at least one gait cycle. The controller is configured to perform a classification of the kinematic data and EMG recordings in accordance with a subject-specific algorithm, as well as predict the subject's intent to move or prepare for movement in accordance with the rigid (R) terrain scenario or the transition (T) terrain scenario based on the classification of the kinematic data and EMG recordings. The controller is also configured to dynamically modify, in real-time, the one or more control settings of the control device in response to the classification of the kinematic data and EMG recordings.

Another aspect of the invention relates to a powered locomotion assistance system comprising the electronic control system as described herein above, and at least one powered lower limb prosthesis attached to a compromised limb or to a connection point corresponding to a missing limb of a subject. Still other aspects of the invention include methods of providing locomotion assistance to a patient in need of such assistance, including attaching the various elements of the powered locomotion assistance system as described herein to a subject and operating the controller as described herein.

### BRIEF DESCRIPTION OF THE DRAWINGS

The foregoing summary and the following description will be better appreciated and understood in conjunction with the non-limiting examples illustrated in the attached drawing figures, of which:

FIG. 1 is a schematic diagram depicting an exemplary electronic control system for a powered lower limb prosthesis in accordance with aspects of the invention;

FIG. 2 is a schematic diagram depicting an exemplary gait cycle in accordance with aspects of the invention;

FIG. 3A depicts a sequence of modifications of a range of stiffness levels during a gate cycle in accordance with clinical testing of the exemplary electronic control system of FIG. 1;

FIG. 3B is a photograph depicting a subject on a Variable Stiffness Treadmill (VST) platform in accordance with clinical testing of the exemplary electronic control system of FIG. 1;

FIG. 4 is a schematic representation of continuous overlapping window segmentation on filtered electromyographic (EMG) recordings during a gait cycle in accordance with clinical testing of the exemplary electronic control system of FIG. 1;

FIG. 5 depicts a selection methodology for selecting optimum features extracted from kinematic data and EMG recordings in accordance with aspects of the invention;

FIG. 6 depicts a heatmap of appearance frequency of the selected EMG features per muscle variable in accordance with clinical testing of the exemplary electronic control system of FIG. 1;

FIG. 7 depicts a heatmap of appearance frequency of the selected kinematic features per joint variable in accordance with clinical testing of the exemplary electronic control system of FIG. 1;

FIG. 8A depicts a performance evaluation of utilizing k-NN window classifiers across all subjects in accordance with clinical testing of the exemplary electronic control system of FIG. 1;

FIG. 8B depicts a performance evaluation of utilizing an Artificial Neural Network (ANN) algorithm across all subjects in accordance with clinical testing of the exemplary electronic control system of FIG. 1;

FIG. 9 depicts a table of features extracted from the EMG signals in accordance with aspects of the invention; and

FIG. 10 depicts algorithmic implementation of a Particle Swarm Optimization (PSO) methodology for feature selection in accordance with aspects of the invention.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

In the following detailed description, numerous specific details are set forth by way of examples in order to provide a thorough understanding of the relevant design features. However, it should be apparent to those skilled in the art that the present design features may be practiced without such details. In other instances, well known methods, procedures, components, and circuitry have been described at a relatively high-level, without detail, in order to avoid unnecessarily obscuring aspects of the present design.

Additionally, various forms and embodiments of the invention are illustrated in the figures. It will be appreciated that the combination and arrangement of some or all features of any of the embodiments with other embodiments is specifically contemplated herein. Accordingly, this detailed disclosure expressly includes the specific embodiments illustrated herein, combinations and sub-combinations of features of the illustrated embodiments, and variations of the illustrated embodiments.

Terms concerning attachments, coupling and the like, such as “connection,” “mounted,” “connected” and “interconnected,” refer to a relationship wherein structures are secured or attached to one another either directly or indirectly through intervening structures, as well as both movable or rigid attachments or relationships, unless expressly described otherwise.

In general, aspects of the invention relate to systems and methods for providing a subject-specific pattern recognition and classification strategy using kinematic data and surface electromyographic (EMG) signals to recognize user intent to transition from a rigid to a compliant surface. The systems and methods described herein can accurately predict upcoming surface stiffness transitions in time to allow for fast parameter control of the prostheses and for adaptation to the new terrain. The proposed framework can lead to increased robustness and safety of lower-limb prostheses that will eventually improve the quality of life of individuals living with a lower limb amputation.

Aspects of the invention are applicable not only to applications for powered ankle-foot prostheses, but also in the area of lower limb prostheses, orthotic devices, and any type of controlled braces or (e.g. lower-limb) exoskeletons, among others. Accordingly, to the extent aspects of the disclosure herein refer to control of a “lower limb prosthesis,” it should be understood that any of the techniques applied thereto may also be applied to analogous portions of a controlled orthotic, brace, or exoskeleton (sometimes referred to as a “robotic exoskeleton”), in particular, a lower limb assistive device or portions thereof. The term “prosthesis” is thus used herein to refer to any combination of elements that provides controlled ambulation assistance to a wearer, including but not limited to entirely artificial body parts intended to replace an amputated limb as well as structures intended to be attached to an existing, compromised limb. The term “subject” is used herein to refer to the wearer or use of such an ambulation assistance system. The term “lower limb” is thus not limited to human legs but may refer to any portion of a subject that provides a supportive or ambulatory force relative to the surface on which the subject is ambulating. Further, to the extent the disclosure refers to “muscles” it should be understood that this term is not limited to natural muscle tissue, but also to artificial muscles or other non-living components of a exoskeleton, or prosthesis that perform functions analogous to muscles (e.g. contracting or expanding to provide motive forces of a skeletal form to which it is attached). Finally, although the control system is described relating to a biped, the invention is not limited to a subject having any particular number of legs. Referring generally to the figures, an electronic control system 100 for a powered lower limb prosthesis configured to be attached to a connection point of a subject 200 is illustrated. In one example, the subject 200 is a human amputee having an amputated or leading leg 120 (to which a lower limb prosthesis 160 may be attached) and a non-amputated or trailing leg 110. Thus, in a subject-amputee, the powered lower limb prosthesis 160 (discussed below) comprises a lower limb prosthesis attached to a connection point corresponding to the amputated leg 120. In a subject with no amputations (e.g. one more compromised limbs which may be non-functional or non-fully-functional), the lower limb prosthesis may attach to the limb or portions thereof to provide sufficient control of the compromised limb.

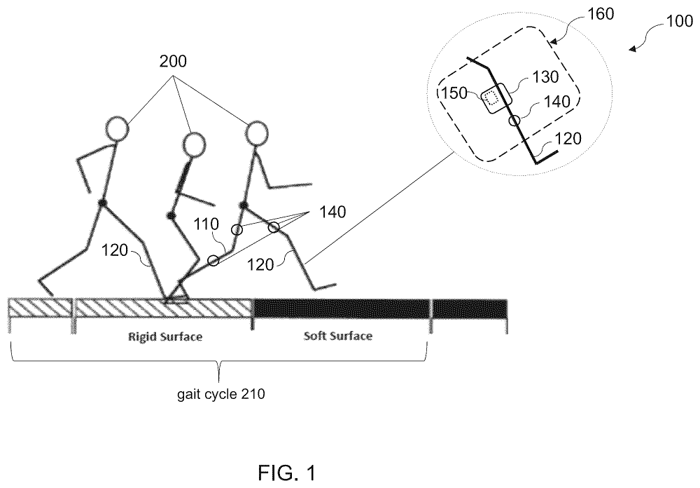

More specifically, FIG. 1 is a schematic diagram depicting an exemplary electronic control system 100 for a powered lower limb prosthesis 160 in accordance with aspects of the invention. In one non-limiting example, the lower limb prosthesis 160 includes an ankle joint. In another non-limiting example, the lower limb prosthesis 160 is a powered ankle-foot prosthesis.

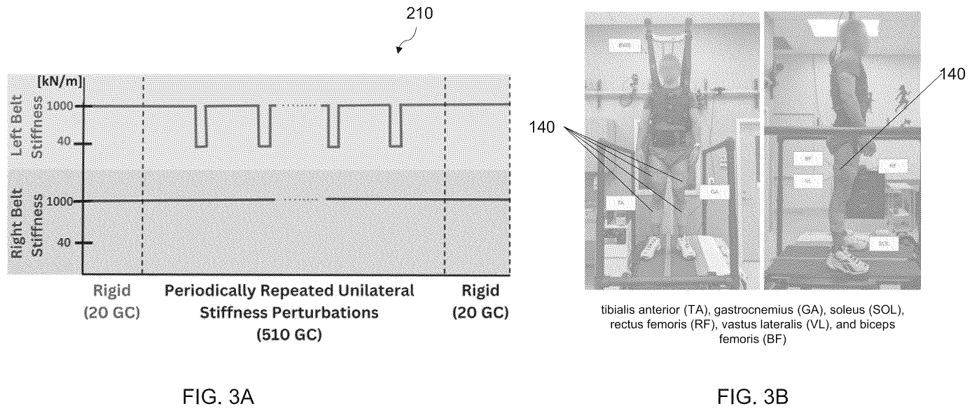

The control system 100 includes a control device 130 for one or more joints of the lower limb prosthesis 160, as well as a plurality of sensors 140 attached to the subject 200. One skilled in the art would understand from the description herein that the placement of the plurality of sensors 140 as illustrated in FIGS. 1 and 3B is not intended to be limiting. Rather, the placement of the plurality of sensors 140 may be determined, in part, by the collected kinematic data (e.g., data related to the motion of one or more of a hip joint, a knee joint, and an ankle joint of the subject 200 during the at least one gait cycle 210 performed by the subject 200). Similarly, placement of the control device 130 is not limited to what is illustrated for example in FIGS. 1 and 3, and can be determined, in part, by the type of lower limb 160 is used, kinematic data to be collected from a hip joint, a knee joint, and an ankle joint of the subject 200, or a combination thereof.

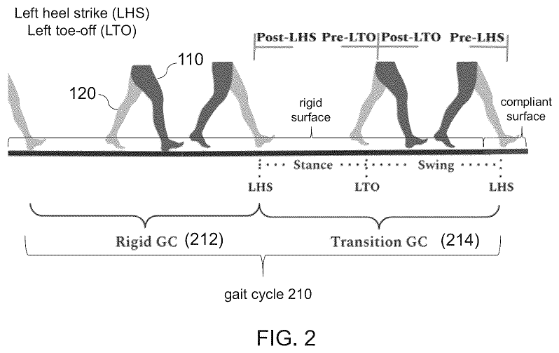

The plurality of sensors 140 is and configured to capture kinematic data and electromyographic (EMG) recordings during the at least one gait cycle 210 performed by the subject 200. FIG. 2 is a schematic diagram depicting an exemplary gait cycle in accordance with aspects of the invention. In an exemplary embodiment, the at least one gait cycle begins and ends at Left Heel Strike (LHS), with subject's leading (amputated leg) being the left leg. Additionally, the at least one gait cycle 210 comprises at least one of: a rigid (R) terrain scenario 212 corresponding to movement or preparation for movement of the subject 200 between a rigid surface and another rigid surface, and a transition (T) terrain scenario 214 corresponding to movement or preparation for movement of the subject 200 between the rigid surface and a non-rigid or compliant surface.

The control system 100 also includes a controller 150 coupled to the control device 130. The controller 150 is configured to control one or more control settings of the control device 130 during at least one gait cycle 210 performed by the subject 200 in accordance with instructions stored in a digital memory. In an exemplary embodiment, the controller 150 is configured to process the kinematic data related to motion of the subject 200. In a non-limiting example, the kinematic data includes data related to the motion of one or more of a hip joint, a knee joint, and an ankle joint of the subject 200 during the at least one gait cycle 210 performed by the subject 200. Additionally, the controller 150 is configured to process the surface EMG recordings of one or more of the subject's 200 muscles during the at least one gait cycle 210. In one non-limiting example, the one or more of the subject's 200 muscles includes six muscles, such as a tibialis anterior (TA), a vastus lateralis (VL), a biceps femoris (BF), and a rectus femoris (RF) of the subject's 200 amputated leg 120. Additionally or optionally, the subject's 200 muscles includes a soleus (SOL) and a gastrocnemius (GA) of the subject's non-amputated leg 110.

Still further, the controller 150 is configured to perform a classification, such as a binary classification, of the kinematic data and EMG recordings in accordance with a subject-specific algorithm. In one non-limiting example, the controller 150 is configured to perform a binary classification as corresponding to the rigid (R) terrain scenario 212 or the transition (T) terrain scenario 214. Additionally or optionally, the controller 150 is configured to perform a multiclass classification. In one embodiment, the multiclass classification includes the transition (T) terrain scenario 214 having a plurality of levels or degrees of compliance, as characterized, for example, by perceptible softness of the surface. Based on this classification of the kinematic data and EMG recordings, the controller 150 is configured to predict the subject's intent to move or prepare for movement in accordance with the rigid (R) terrain scenario 212 or the transition (T) terrain scenario 214. Further, the controller 150 is configured to dynamically modify, in real-time, the one or more control settings of the control device 130 in response to the classification of the kinematic data and EMG recordings.

Additionally or optionally, the controller 150 is configured to process the EMG recordings of the subject's leading (amputated leg 120) hip muscles during the at least one gait cycle 210. Thus, the subject's leading hip muscles include the VL, the BF, and the RF muscles. The controller 150 is configured to process the EMG recordings of the subject's trailing (non-amputated leg 110) muscles during the at least one gait cycle 210. Thus, the subject's trailing muscles comprising the SOL and the GA muscles. The controller 150 is further configured to process the kinematic data of the subject's leading (amputated leg 120) and trailing (non-amputated leg 110) during the at least one gait cycle 210. In this way, the controller 150 is configured to process the kinematic data related to a flexion characteristic, extension characteristic, or velocity of the ankle joint, the knee joint, the hip joint, or a combination thereof of the subject 200.

The controller 150 is configured to perform a classification, such as a binary classification, of the EMG recordings and kinematic data as corresponding to the rigid (R) terrain scenario 212 or the transition (T) terrain scenario 214 using a k-Nearest Neighbors (k-NN) classifier. In this way, the controller 150 predicts the subject's 200 intent to move or prepare for movement in accordance with the R terrain scenario 212 or the T terrain scenario 214 based on the classification of the EMG recordings and kinematic data. Additionally or optionally, the controller 150 then dynamically modifies, in real-time, the one or more control settings of the control device 130 in response to the classification of the classification of the EMG recordings and kinematic data. In an exemplary embodiment, the one or more control settings comprises a flexion command, an extension command, or a combination thereof, to be applied to the one or more joints. This dynamic modification permits individuals with gait impairment generally and more specifically, wearers of lower limb devices or lower limb prostheses to ambulate smoothly and adapt to changing environments (e.g. on non-flat and soft or compliant surfaces).

Additionally or optionally, the controller 150 is configured to extract, in real-time, a plurality of features from the kinematic data and EMG recordings. From these extracted plurality of features, the controller 150 is configured to apply, in real-time, a selection methodology to select optimum features. In an exemplary embodiment, the selection methodology is a Particle Swarm Optimization (PSO) method. The controller 150 is configured to then utilize an Artificial Neural Network (ANN) algorithm to predict the subject's 200 intent to move or prepare for movement in accordance with the R terrain scenario 212 or the T terrain scenario 214. Thus, when the controller 150 is configured to perform one or more actions in real-time, the controller 150 is configured to perform the one or more actions instantaneously or without human-perceptible delay. In an exemplary embodiment, when the controller 150 is configured to perform one or more actions in real-time, the controller 150 is configured to perform the one or more actions for a duration that is no longer than an elapsed time between an input time and an output time. The input time includes a time when the kinematic data, surface EMG recordings, or combination thereof is received by the controller 150, for example, and the output time is another time when the one or more control settings of the control device 130 must be modified for the subject 200 to achieve an improvement in gait, e.g. a smoother gait. The smoother gait requires the subject 200 to ambulate smoothly or adapt to movement in accordance with the R terrain scenario 212 or the T terrain scenario 214.

As used herein, the term “smoothness” refers to the degree to which gait characteristics are defined by a lack (or minimization) of pauses or perceptible increases or decreases in speed of motion and/or defined by an efficiency of movement (e.g. of the lower extremity or portions thereof) from a first point to a second point along the gait cycle along a gradual path with a lack or minimization of perceptibly sudden inflections and/or falls. Thus, as is known in the art, one measure of gait smoothness uses harmonic ratios (HR)—the ratios between the sum of the magnitudes of the even to the odd harmonics over a single stride—to measure smoothness of gait. Another known measure is based upon the spectral arc length (SPARC)—a measure of the arc length of the Fourier magnitude spectrum within an adaptive frequency range, which is known for quantifying movement intermittencies independent of amplitude or duration. The invention is not limited to the use of these or any particular measurements for characterizing smoothness, and discussion herein of improving smoothness may include characterization of improvements by any method generally accepted by those of skill in the art as being objective, definite, repeatable, and/or predictable.

### EXAMPLE

The co-inventors assessed the exemplary systems as disclosed herein in a laboratory setting, to validate feasibility and functionality of the components of the subject systems, as well as verified any updates or improvements made.

Objective: A subject-specific, pattern recognition (PR) and classification strategy using kinematic data and EMG signals from several muscles of both lower limbs to create an advanced, high-level control device (e.g. control device 130) for lower limb prostheses (e.g. powered ankle-foot prostheses) capable of recognizing user intent to transition from a rigid surface to another rigid surface (e.g. R terrain scenario 212) and between a rigid surface and a compliant surface (e.g. T terrain scenario 214). Thus, wearers of lower limb prostheses 160 with control device 130 achieves improvements in terms of a natural and robust ambulation over non-rigid surfaces. This is achieved, in part, by combining surface EMG signals and kinematic data with a phase-dependent PR algorithm for identifying cases of traversing rigid and compliant terrains.

### Experiment Protocol

To investigate the mechanisms of human locomotion during ambulation in dynamic environments, eight subjects (age 26.6±2.2 years, height 174.2±8.7 cm, mass 70.8±12.3 kg) who were free from any orthopedic or neurological pathology participated in the study. As shown in FIGS. 3A-3B, all participants were asked to walk on the Variable Stiffness Treadmill (VST), which is a split-belt treadmill with a walking surface that can interactively and dynamically change its vertical compliance. Specifically, one side of the treadmill is able to lower its stiffness to simulate walking on a soft surface. The VST permits control over a wide range of stiffness levels that can be modified during the gait cycle (e.g. gait cycle 210) and by extension allows the induction of multiple force perturbations to the subject's legs. In the study, the right side of the treadmill remained rigid for the entire duration of the experiment, while expected stiffness perturbations were applied unilaterally to the leading (left) leg. The change in the stiffness of one belt was based on the assumption that transitions between surfaces in everyday life activities are first experienced by one leg. For the purposes of this study, the leading leg transitioning between surfaces was the left leg. As shown in FIG. 3A, the sequence of the changes in surface stiffness for the leading or left leg was: 20 sets of 1 rigid surface cycle (excluded from any data processing), 51 sets of 9 rigid surface cycles followed by 1 compliant surface cycle (resulting in 510 gait cycles), and finally 20 sets of 1 rigid surface cycle. The right leg was always on rigid ground. Further, the stiffness on the left side of the treadmill dropped from 1000 kN/m, which simulates rigid ground, to 40 kN/m, which simulates a compliant surface (e.g., feels like walking on a surface such as sand or grass). The stiffness perturbations occurred periodically every ten rigid gait cycles and all subjects were verbally informed as early as three steps before an upcoming perturbation. The timing of each ground stiffness change ensured that the left leg was always experiencing a walking surface with a stiffness of 40 kN/m on a perturbed gait cycle.

Before data collection began, the subjects were given the chance to choose a comfortable walking speed. Different treadmill speeds between 70 and 100 cm/s were tested until each subject found a suitable pace that closely resembled their normal, everyday walking patterns. For all 8 subjects, the chosen speed was 90 cm/s. In total, each subject walked on the VST for 554 gait cycles, which corresponds to approximately 12 minutes. The purpose of the first 20 gait cycles was to introduce the subjects to walking on our treadmill and familiarize them with the setup. Data pertaining to these gait cycles were omitted from any further processing. A body harness that did not offset any of the subjects' weight was used during all experiments, solely for safety purposes (FIG. 3B).

Kinematics: Each subject was instrumented with a plurality of sensors 140, such as 23 reflective motion capture markers attached to their pelvis, thighs, shanks, feet, spine, and torso. The markers were used for tracking the motion of the subjects' legs and for extracting upper body parameters. Kinematic data for both legs were obtained at 100 Hz using a motion capture system (e.g. as designed by Vicon Motion Systems Ltd) that is integrated with the VST. Kinematic data was utilized for timing the changes in the treadmill surface stiffness with the subject motion.

Electromyography: The muscle activity of both legs was obtained using the plurality of sensors 140, such as twelve wireless surface EMG electrodes (e.g. Trigno® sensors as designed by Delsys Inc.). The surface electrodes were placed on six major muscles of the lower limbs following SENIAM (surface EMG for a non-invasive assessment of muscles) recommendations. The monitored muscles included the tibialis anterior (TA), gastrocnemius (GA), soleus (SOL), rectus femoris (RF), vastus lateralis (VL), and biceps femoris (BF) (FIG. 3B). The selection of the muscles was determined, in part, based on their primary role in ankle motion and stability, in which the TA produces dorsiflexion of the foot, while the GA and SOL muscles produce plantar flexion of the foot. In the absence of ankle plantar flexor power, as seen in transtibial amputees, hip extensors and flexors, as well as hip external rotators have been shown to become the major power generators. The RF, BF, and VL muscles were thus selected to study how knee and hip EMG signals contribute to identifying transitions from rigid to compliant surfaces. The EMG signals were sampled at 2 kHz and were synchronized with the motion capture data. The experimental protocol was approved by the University of Delaware Institutional Review Board (IRB ID #1544521-7) and informed consent from the subjects was obtained at the time of the experiment.

### Data Processing

The raw kinematic and muscular activity data were synchronized by utilizing the real-time Foot VErtical & Sagittal Position Algorithm (F-VESPA) for heel-strike detection (described in C. Karakasis and P. Artemiadis, “F-VESPA: A Kinematic-based Algorithm for Real-time Heel-strike Detection During Walking,” 2021 IEEE/RSJ International Conference on Intelligent Robots and Systems (IROS), Prague, Czech Republic, 2021, pp. 5098-5103, incorporated herein by reference). Heel-strike detection allowed for the data to be segmented into gait cycles following the convention that each gait cycle 210 begins and ends at Left Heel Strike (LHS), as illustrated in FIG. 2. Outlier gait cycles including sensor noise or other artifacts in the recorded data were identified after the application of an outlier detection method for periodic data, as described for example in B. Hobbs and P. Artemiadis, “A Systematic Method for Outlier Detection in Human Gait Data,” International Conference on Rehabilitation Robotics (ICORR), 2022, incorporated herein by reference in its entirety for all purposes. On average a total of 9±2 outlier gait cycles were found per subject and excluded from the total data.

EMG raw data was first filtered with a 4th-order Butterworth band-pass filter with low and high frequencies at 50 Hz and 500 Hz, respectively. The data was then full-wave rectified. The linear envelope of the signal was computed and the data was filtered with a 4th-order Butterworth low-pass filter with a 4 Hz cut-off frequency. The final processed EMG data were normalized to the maximum value of each muscle during the experiment. Temporal normalization of EMG data is necessary given that muscle activity during walking is highly dependent on the phase of the gait cycle. Therefore, the data was also temporally normalized to percent gait cycle, where 0% and 100% correspond to the heel strike of the left leg at two successive gait cycles. The EMG recordings acquired during rigid surface walking were separated from the gait cycles before, during, and after each compliant surface transition. Each gait cycle was specifically labeled as either i) rigid (R) if the subject was walking on a rigid surface preparing to step on a rigid surface (e.g. rigid terrain scenario 212), ii) transition (T) if the subject was walking on a rigid surface preparing to step on a compliant surface, iii) perturbation (PT) if the subject was walking on a compliant surface, iv) recovery (RC) if the subject was walking on a rigid surface following a perturbation (as illustrated in FIG. 2). The PT and RC data were excluded from the data analysis and we focused on the comparison between the R and T cases, which pertain to the anticipatory component of our study.

### Data Analysis

### Continuous Window Segmentation

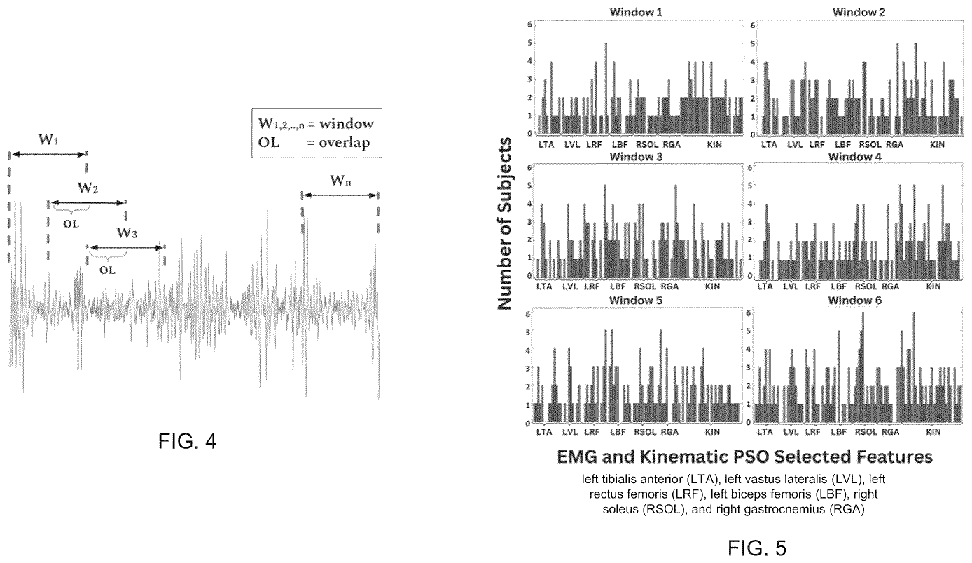

EMG signals are time-varying. Specifically, EMG recordings from the lower limbs show large variations within the same gait class. Therefore, utilizing the data of an entire gait cycle as input to a classification algorithm might result in overlaps of features among classes, and therefore, low PR accuracy. In real-time applications, this approach is inadequate for safe and robust prosthetic control. For this reason, a sliding window approach (see FIG. 4) as used, such that the EMG signals were segmented into windows ranging from 50 to 250 ms. Within these time windows, EMG signals can be modeled as stationary and subsequently provide features for a reliable PR design. In particular, data analysis was performed with a window duration of 150 ms. A 50% window overlap between the segments was chosen for continuous feature extraction. Since some natural variability in the gait is expected, the number of samples in each gait cycle varies per subject. Re-samples of the EMG and kinematic data were obtained and an average number of samples was observed in each subject prior to applying windowing segmentation. This ensures a uniform number of samples per window for all gait cycles within the same subject.

Statistically significant changes (using a t-test) in EMG activation and kinematics were observed mainly during the terminal stance and swing phases. Thus, focus of the analysis included data from the Pre-LTO (55% of gait cycle (GC)) up to the Pre-LHS (100% of GC) phases of each gait cycle (see FIG. 2). This observation is aligned with the hypothesis that the prediction accuracy of the classifier will be higher toward the end of each gait cycle, as the subjects approach the compliant surface. The window segmentation was applied exclusively on the 55-100% of the gait cycle, resulting in overall six overlapping analysis windows. The following analysis steps were done separately for each of the segmented windows.

### Feature Extraction and Selection

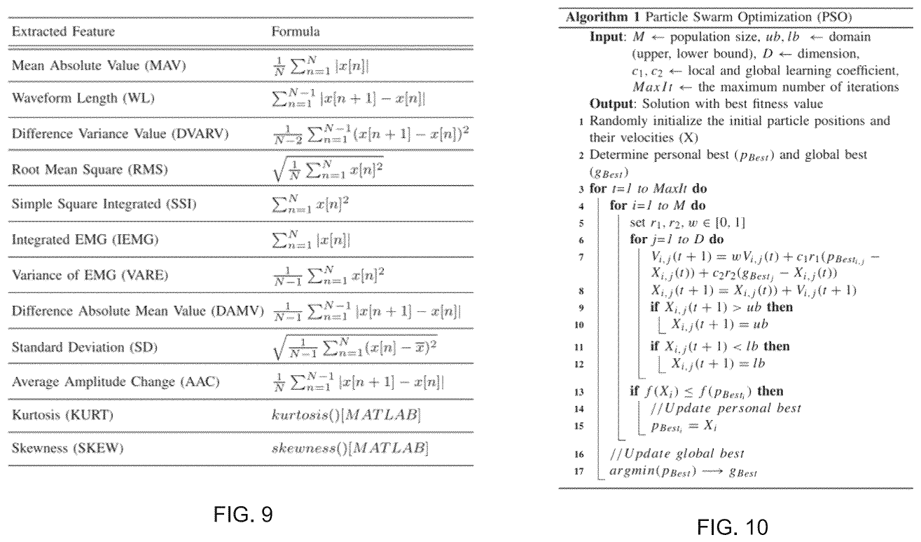

Feature extraction and selection are important steps in achieving optimal classification performance. The feature classification can be carried out in the time domain (TD), frequency domain (FD) and time-frequency domain (TFD). Due to their implementation and computational simplicity, TD features are frequently used in pattern recognition as they satisfy the requirement of fast time-response. For the electromyographic data, six muscles were studied: the TA, VL, BF, and RF of the left leg, and the SOL, and GA of the right leg. The other muscles were excluded from the analysis, because of the lack of activation or lack of statistically significant differences between the R and T cases during the Pre-LTO and Pre-LHS phases of each GC. 12 EMG features were extracted, according to their prominent use in the literature, as a non-exhaustive compilation of myoelectric signal features employed in pattern recognition for prosthetic control (as shown in the table illustrated in FIG. 9). FIG. 9 depicts a table showing Set of TD features chosen to be extracted from the EMG signals along with their formulas or functions used for their calculation. Further, in the table shown in FIG. 9, x[n] represents the pre-processed EMG signal sample at discrete time n, where n=0, 1, 2, . . . , N, where N is the number of samples in a specific segment. A total of 72 EMG features were extracted per segmented window, which correspond to 12 features for each of the 6 muscles used. For the kinematic data, the flexion/extension of the hip, knee, and ankle of both legs, as well as the velocity of the latter two joints were studied. The maximum, mean, and minimum values of these kinematic variables were extracted, resulting in a total of 30 kinematic features per segmented window.

### Particle Swarm Optimization (PSO)

After feature extraction, the result is a 102 (features)×6 (windows)=612 elements wide feature vector per GC and subject. High-dimensional feature vectors lead to information redundancy, which results in a decrease in classification accuracy and an increase in computational complexity. Thus, the dimension of the feature input vector is reduced and a feature selection method that preserves the important characteristics of the dataset, but decreases complexity is applied. The PSO algorithm is used to select a subset of relevant features for use in model construction, in order to make predictions faster and more accurate. PSO is an algorithm influenced by the habit of bird flocking or fish schooling used for generating an optimal number of features to be used for a certain task like classification.

The PSO algorithm searches in the space of an objective function by adjusting the trajectories of individual particles in a quasi-stochastic manner. Each particle, combining its own best experience in the history of the search with the global best solution of the swarm, adjusts its velocity and position (as shown in Algorithm 1 depicted in FIG. 10). The PSO parameters were initialized according to values widely used in literature and the final values were optimized for our problem. According to existing literature, selecting a population size between 10-30 renders optimal results. After cross-validation, the value M=30 was selected as it provided both fast and accurate algorithm convergence. The acceleration constants c1, c2 define the ability of the group to be influenced by the best local (particle personal) solutions found over the iterations and the ability of the group to be influenced by the best global solution found over the iterations respectively. We chose c1=c2=2 to allow for equal exploration and exploitation dynamics. The maximum number of iterations was set to MaxIt=100. As an optimization method, PSO takes all of the existing features into account, runs an optimization scheme in order to minimize or maximize a cost function and it finds the combination of features that achieves this goal. For the current study, the objective function is formed as a combination of two aims: (i) maximizing the classification accuracy of the classifier, (ii) minimizing the number of features that the PSO selects.

### Classification

The hypothesis of this study is a classifier can be trained to differentiate between rigid and compliant surface transitions based on a fusion of neural and kinematic features. The present study employs the k-Nearest Neighbors (k-NN) method to identify and classify walking patterns on transitions from rigid-to-rigid surfaces and from rigid-to-compliant surfaces using kinematic and EMG data from both lower limbs. The data can thus be separated into two classes, which correspond to a step between (1) rigid-rigid surface (R) or a rigid terrain scenario 212, and (2) rigid-compliant surface (T) or a transition terrain scenario 214. Classifiers, such as a binary classifiers, are therefore developed to separate data into one or more classes.

### Data Augmentation

The extracted data and features discussed above was split into the training and testing set following the 70/30 rule. Due to the experiment protocol (see FIG. 3A), the R cases were significantly more (approx. 90%-majority class) than the T cases (approx. 10%-minority class), leading to an imbalanced classification problem. In order to minimize the effect of the data imbalance, an oversampling technique was applied to the training set and an undersampling technique in the test set. The Synthetic Minority Oversampling TEchnique (SMOTE) was employed to increase the number of T cases relative to the R cases during the training of the classifier and decrease the bias towards the majority class in the data. SMOTE oversamples the minority class by creating “synthetic” examples rather than by over-sampling with replacement. The minority class is over-sampled by taking each minority class sample and introducing synthetic examples along the line segments joining any/all of the k′ minority class nearest neighbors. Depending on the amount of over-sampling required, neighbors from the k′ nearest neighbors are randomly chosen. In this study, implementation currently uses k′=5 nearest neighbors. A random R case undersampling was applied to balance the testing data set to 50% R-50% T, and thus enable the use of common metrics for classifier performance evaluation. Undersampling does not insert any type of bias in the evaluation of the classifier.

k-NN Algorithm

The The k-NN algorithm is a supervised learning classifier, which uses proximity to make classifications or predictions about the grouping of an individual data point. A class label is assigned on the basis of a majority vote from the k nearest neighbors of the specified data point. The label that is most frequently represented around the neighborhood of a given data point is used. In order to determine which data points are closest to a given query point, distance metrics were used to form decision boundaries and partition query points into different regions. This implementation utilizes Euclidean distance d between a test and a training data point, in combination with an inverse distance weight (w=1).

The k value in the k-NN algorithm defines how many neighbors will be checked to determine the classification of a specific query point. In this study, k=9 as optimal for the extracted dataset after running trials with a series of different k values and cross-validating this choice. For algorithm implementation, the built-in Matlab (Mathworks) function fitcknn( ) was used. Each predictor variable was centered and scaled by the corresponding weighted column mean and standard deviation. For each segmented window, a separate classifier was trained, resulting in overall six separate classifiers and decisions per gait cycle.

### Artificial Neural Network (ANN)

A pattern recognition neural network (NN) was developed to deduce a final decision per gait cycle, by combining the individual six decisions of the window classifiers. The network implementation was based on the built-in Matlab (Mathworks) function patternnet( ) with 1 hidden layer of 100 nodes. Using the trained window k-NN classifiers, the existing training data was used to extract a binary decision per window for each gait cycle. The binary decisions were then fed to the ANN for training purposes. The testing data that were used to evaluate the window classifiers were used for evaluating the ANN performance as well.

The pattern recognition network was applied serially in consecutive combinations of the window classifiers beginning from data only from window 1, continuing to data from windows 1 and 2, 1 and 2 and 3, leading up to all six windows. The window combinations are tested to evaluate how fast within the gait cycle the applied algorithm can make a decision regarding the walking surface of the next step. The pattern recognition network serially combines the information of the independent k-NN classifier windows in an effort to evaluate how many windows are necessary to make an accurate prediction between the R and T cases. The number of the k-NN windows included in each combination defines the speed of the prediction, with combination 1 being the fastest, as there is only information from the first window classifier involved, and combination 1+2+3+4+5+6 being the slowest case, as the algorithm needs information from all windows to reach a decision. Adding the ANN aims in developing a robust, accurate, and prompt real-time user intent recognition framework. Real-time classification requires a decision to be made prior to LHS and therefore before stepping on the new surface, either rigid or compliant. The hypothesis is that a serial combination of the distinct classifier decisions will contribute to higher accuracy and faster predictions.

### Performance Evaluation—Classification Metrics

In order to evaluate the performance of the classification algorithm, a series of metrics that are based on the structure of the confusion matrix is defined. A confusion matrix is a tabular visualization of the ground-truth labels versus model predictions. Each row of the confusion matrix represents the instances in a predicted class and each column represents the instances in an actual class. In this confusion matrix structure, True Positive (TP) represents how many positive class samples the model predicted correctly. True Negative (TN) represents how many negative class samples the model predicted correctly. False Positive (FP) represents how many negative class samples the model predicted incorrectly. False Negative (FN) represents how many positive class samples the model predicted incorrectly. The R cases were designated as the positive class and the T cases were designated as the negative class. The metrics that were chosen to evaluate our model are as follows:
Precision: It measures how many observations predicted as R are in fact R.

$$P = \frac{T P}{T P + F P} \qquad ( Equation 1 )$$

Sensitivity: It measures how many observations out of all R observations have been classified as R.

$$S = \frac{T P}{T P + F N} \qquad ( Equation 2 )$$

Specificity: It measures how many observations out of all T observations have been classified as T.

$$S P = \frac{T N}{T N + F P} \qquad ( Equation 3 )$$

F1-score: It is the harmonic mean between precision and sensitivity.

$$F 1 = \frac{2 \cdot TP}{2 \cdot TP + FN + FP} \qquad ( Equation 4 )$$

Balanced Accuracy: It is the arithmetic mean of sensitivity and specificity. This metric was chosen over simple Accuracy to eliminate any bias on performance evaluation due to the data imbalance.

$$BalAcc = \frac{1}{2} ( S + SP ) \qquad ( Equation 5 )$$

Balanced Accuracy and F1-score, as they combine the other metrics well, were chosen as representative quantities to measure average subject-specific classification performance.

### Results

### Feature Extraction and Selection

The PSO algorithm was able to reduce the dimensionality of the input feature vector by 75% on average. Specifically, the initial 102 features per window were reduced to approximately 22±5 across subjects. The variation in the number of selected features is due to the fact that each window corresponds to a different classifier, while a natural variability between the subjects' features is also to be expected. Analyzing frequency of appearance of each feature across windows and subjects could result in useful conclusions regarding the features that are more informative and distinct between the two analyzed cases. Results regarding the frequency of the features across windows are shown in FIG. 5 (PSO selected features averaged for all subjects per window classifier. EMG features are grouped per muscle. Each muscle group includes 12 features (see Table in FIG. 9). Kinematic features are grouped together). Specifically, some features were chosen by the PSO consistently for more than 1 subject. For example, in window 6, there are two features that were selected for six out of the eight study participants. This strongly indicates that besides the subject-specific algorithm developed in this study, a generalized pattern recognition strategy is feasible by extracting a set of features appearing consistently in the majority of the subjects.

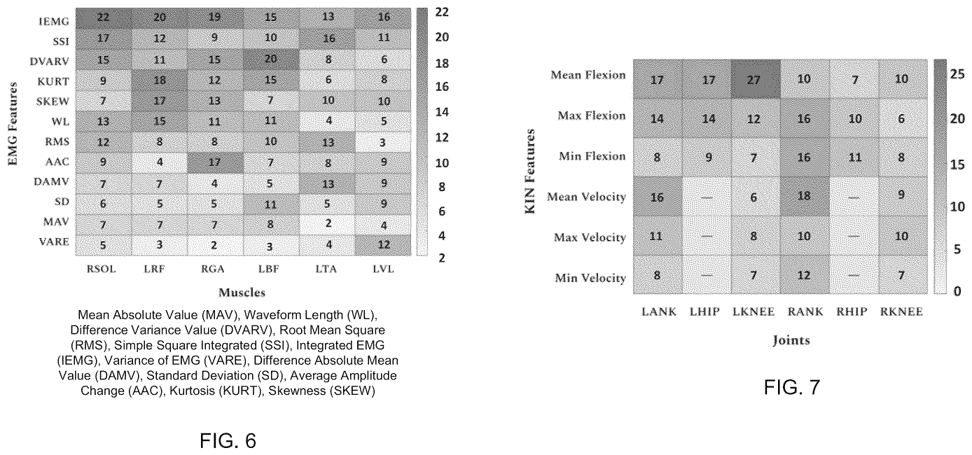

Moreover, the selected features are ranked in descending order according to their selection frequency among the subjects in FIGS. 6 and 7, showing EMG and kinematic features respectively. In particular, FIG. 6 shows a heatmap of appearance frequency of the selected EMG features per muscle variable. As an example, the IEMG feature from RSOL appears in 2 subjects in Windows 1 and 5 (4 times), in 4 subjects in Windows 2, 3 and 4 (12 times), and in 6 subjects in Window 6 (6 times), totaling 22 times across all subjects and windows. FIG. 7 shows a heatmap of appearance frequency of the selected kinematic features per joint variable. As an example, the mean flexion of LANK appears in 2 subjects in Windows 1 and 3 (4 times), in 4 subjects in Windows 2 and 4 (8 times), and in 5 subjects in Window 6 (5 times), totaling 17 times across all subjects and windows. Overall, the results show that the number of features could significantly be reduced without losing generality across subjects. This is important because selecting a subset of the features contributes to reducing the channels of information that are used per experiment, increasing computational efficiency and facilitating the classification process.

Regarding the available channels of information for the case of lower-limb amputees, the fact that we can further reduce the set of features computed per muscle is very promising. The chosen group of muscles used for classification consists of generally bigger muscles parts of which are highly likely to be found as residual muscles in subjects with transtibial amputations. Assuming that the leading (left) leg is the amputated limb, it is very exciting to note that the EMG features are all derived from intact hip muscles (LRF, LBF, LVL). On the contrary, signals from below the knee (RSOL, RGA) are extracted only for the trailing (right), non-amputated limb. This finding is particularly important since hip muscles become more significant for stabilizing the lower limb during walking for subjects, such as human amputee patients.

### Classification

k-NN

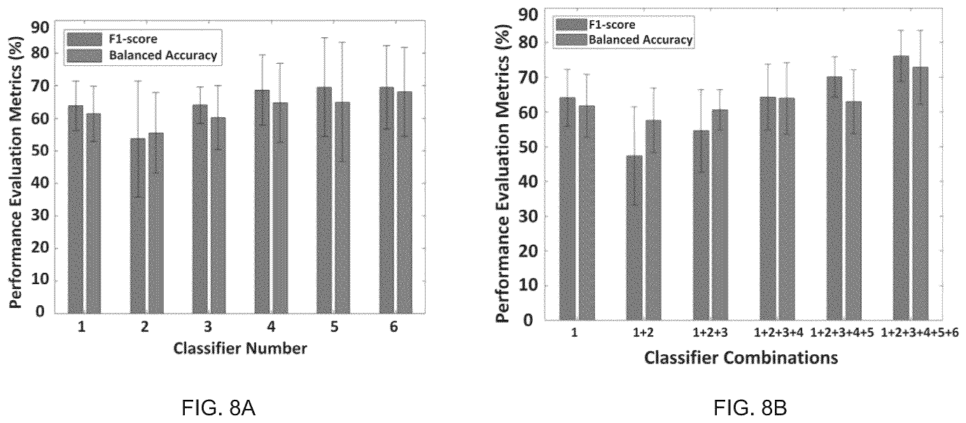

The performance of each window classifier confirms the hypothesis that the prediction accuracy will be higher toward the end of each gait cycle and the last segmented windows. Performance evaluation of the best subject results showed that Balanced Accuracy steadily increased by 41.7% between window 1 (45.8%) and window 6 (87.5%). All the measured metrics presented a similar upwards trend towards the last segmented windows, confirming an improvement in classifier performance as the subject approached the end of each gait cycle. The study of all five metrics ensures that no bias has been introduced due to data imbalance and shows that our results can be accurately quantified with common metrics found in the literature. This trend was also confirmed for all subjects by studying the average subject performance for each window classifier shown in FIG. 8A. The mean subject values for the F1-score and Balanced Accuracy metrics present an ascending trend as the window (classifier) number increases. The standard deviation calculated for each window shows the dispersion of the metric values among the subjects.

### ANN

Applying the ANN methodology to the resulting k-NN window classifiers, the focus shifts to serial combinations of windows rather than single window classifiers. Again, the trend of the ascending metrics is observed and remains while the F1-score and Balanced Accuracy values increase as window classifiers are added (FIG. 8B). An important observation is that the standard deviation calculated per combination is significantly smaller compared to the single window cases. This finding indicates a narrower dispersion of the metric values around the mean and supports the hypothesis of more robust predictions across subjects. Both of the studied metrics also present similar profiles, showing that although the F1-score tends in general to favor the positive class, which indicates the metric closely resembles the balanced accuracy which accounts equally for both the positive and negative classes. Although window combinations 1-4 and 1-5 present on average 8% lower metric performance than combination 1-6, they manage to maintain a similar, stable profile, while maintaining a small standard deviation of values in contrast to the respective single k-NN window classifiers. Thus, there is a trade-off between classification accuracy and fast prediction time. For example, window combinations 1-4 and 1-5 could be utilized to make a prompt decision about user intent, while compromising an average of 8% accuracy. It should be emphasized that since the developed strategy focuses on subject-specific classification, this trade-off will depend on the subject parameters.

Overall, the addition of the ANN increases the classification accuracy by 4.82% and the F1-score by 6.78% between the k-NN classifier 6 and the classifier combination including windows 1 to 6. For reference, a majority vote was also tested against the ANN training to verify whether training a network offers better results than a simplified majority decision. Indeed, although the majority vote (BalAcc=70.42%, F 1=74.03%) technique performs better than the single k-NN classifier 6 (BalAcc=68.03%, F 1=69.36%), it fails to reach the accuracy level of the ANN (BalAcc=72.85%, F 1=76.14%). Besides being less accurate than the ANN, a majority vote also presents challenges relating to the number of windows involved in the selection process. An even number of windows as in our case requires further subject-specific data thresholding in the case of a tie.

The inherent difficulty of the prediction task is that both classes involved in the classification pertain to walking on rigid ground. The differentiating factor is whether the next step happens on a rigid or a compliant surface. The resemblance of the R and T cases makes prediction a challenging task, which the inventive algorithm successfully tackles by achieving a classification accuracy up to 87.5%. This work can be related and compared to studies on locomotion mode predictions (e.g. stair ascend-descend). A conventional representative work teaches the identification of locomotion modes using EMG signals and reports an average prediction accuracy of 94.8±3.7% during the Pre-HS phase using EMG signals from 16 different muscles of the lower limbs. In contrast, the prediction strategy of the inventive control system 100 reaches an average prediction accuracy of 72.85±9.3%, by only using EMG signals from 6 different muscles of the lower limbs. Thus, the inventive control system 100 successfully predicts transitions between surfaces of variable stiffness while reducing the channels of information necessary to do so.

### CONCLUSIONS

Thus, the inventive pattern-recognition algorithm of the control system 100 predicts the user's intent to transition between rigid and compliant surfaces (e.g. rigid terrain scenario 212 and transition terrain scenario 214) via the use of EMG and kinematic data. The control system 100 effectively reduced the number of extracted features by 75% and selected only the relevant predictor variables as input to the classifiers. The selected features not only contributed to increasing classifier performance and minimizing feature redundancy, but were also consistently selected across subjects. This finding is particularly promising for developing a more generalized approach that is not subject-specific. The ANN PR component allows for a robust and fast prediction that does not exclusively depend on a single classifier decision but incorporates past knowledge from within the gait cycle 210 as locomotion progresses. In particular, the classification strategy was able to robustly and accurately predict user intent to step on a rigid (R) or a compliant (T) surface, achieving an accuracy of up to 87.5%.

The classification takes place between two classes that both involve walking on a rigid surface. The fusion of EMG signals with kinematic data was able to distinguish between the two cases solely on the basis of user intent to step on another rigid or compliant surface. The developed framework can be used as part of the high-level controller (e.g. controller 150) of a powered prosthesis 160 that will inform the prosthesis and tune its parameters in real-time. Such implementation can lead to a new generation of prosthetic devices that will incorporate the human wearer in the loop and proactively adjust their control to transition to surfaces of different compliance. The latter will lead to increased robustness and safety of lower-limb prostheses that will eventually improve the quality of life of individuals living with a lower limb amputation.

Although the invention is illustrated and described herein with reference to specific embodiments, the invention is not intended to be limited to the details shown. Rather, various modifications may be made in the details within the scope and range of equivalents of the claims and without departing from the invention.

1. An electronic control system for a powered lower limb prosthesis configured to be attached to a subject, the electronic control system comprising:
a control device for one or more joints of the lower limb prosthesis;
a plurality of sensors attached to the subject and configured to capture kinematic data and electromyographic (EMG) recordings during at least one gait cycle performed by the subject, each gait cycle comprising at least one of: a rigid (R) terrain scenario corresponding to movement or preparation for movement of the subject between a rigid surface and another rigid surface, and a transition (T) terrain scenario corresponding to movement or preparation for movement of the subject between the rigid surface and a non-rigid or compliant surface; and
a controller coupled to the control device, the controller configured to control one or more control settings of the control device during the at least one gait cycle in accordance with instructions stored in a digital memory, the controller configured to:
process the kinematic data related to motion of the subject, the kinematic data comprising data related to the motion of one or more of a hip joint, a knee joint, and an ankle joint of the subject during the at least one gait cycle performed by the subject,
process the EMG recordings of one or more of the subject's muscles during the at least one gait cycle,
perform a classification of the kinematic data and EMG recordings as corresponding to the rigid (R) terrain scenario or the transition (T) terrain scenario in accordance with a subject-specific algorithm,
predict the subject's intent to move or prepare for movement in accordance with the rigid (R) terrain scenario or the transition (T) terrain scenario based on the classification of the kinematic data and EMG recordings, and
dynamically modify, in real-time, the one or more control settings of the control device in response to the classification of the kinematic data and EMG recordings.

2. The electronic control system of claim 2, wherein the subject is a human amputee having one amputated leg and one non-amputated leg, and the lower limb prosthesis is configured to be attached to a connection point corresponding to the amputated leg.

3. The electronic control system of claim 1, wherein the one or more of the subject's muscles comprises six muscles.

4. The electronic control system of claim 1, wherein the one or more of the subject's muscles comprises: a tibialis anterior (TA), a vastus lateralis (VL), a biceps femoris (BF), and a rectus femoris (RF) of the subject's limb to which the lower limb prosthesis is configured to be attached.

5. The electronic control system of claim 2, wherein the subject's muscles comprise: a soleus (SOL) and a gastrocnemius (GA) of the subject's non-amputated leg.

6. The electronic control system of claim 2, wherein the controller is configured to:
process the EMG recordings of the subject's leading (amputated leg) hip muscles during the at least one gait cycle, the subject's leading hip muscles comprising the VL, the BF, and the RF muscles;
process the EMG recordings of the subject's trailing (non-amputated leg) muscles during the at least one gait cycle, the subject's trailing muscles comprising the SOL and the GA muscles;
process the kinematic data of the subject's leading (amputated) leg and trailing (non-amputated) leg during the at least one gait cycle, wherein the kinematic data comprises data related to a flexion characteristic, extension characteristic, or velocity of the ankle joint, the knee joint, the hip joint, or a combination thereof of the subject;
perform a classification of the EMG recordings and kinematic data as corresponding to the rigid (R) terrain scenario or the transition (T) terrain scenario using a k-Nearest Neighbors (k-NN) classifier;
predict the subject's intent to move or prepare for movement in accordance with the R terrain scenario or the T terrain scenario based on the classification of the EMG recordings and kinematic data; and
dynamically modify, in real-time, the one or more control settings of the control device in response to the classification of the classification of the EMG recordings and kinematic data.

7. The electronic control system of claim 1, wherein the lower limb prosthesis includes an ankle joint.

8. The electronic control system of claim 7, wherein the lower limb prosthesis comprises a powered ankle-foot prosthesis.

9. The electronic control system of claim 1, wherein the one or more control settings comprises a flexion command, an extension command, or a combination thereof, to be applied to the one or more joints.

10. The electronic control system of claim 1, wherein the controller is configured to:
extract, in real-time, a plurality of features from the kinematic data and EMG recordings;
apply, in real-time, a selection methodology to select optimum features from the extracted plurality of features, the selection methodology comprising a Particle Swarm Optimization (PSO) method; and
utilize an Artificial Neural Network (ANN) algorithm to predict the subject's intent to move or prepare for movement in accordance with the R terrain scenario or the T terrain scenario.

11. The electronic control system of claim 1- or 2, wherein when the controller is configured to perform one or more actions in real-time, the controller is configured to perform the one or more actions instantaneously or without human-perceptible delay.

12. The electronic control system of claim 1- or 2, wherein when the controller is configured to perform one or more actions in real-time, the controller is configured to perform the one or more actions for a duration that is no longer than an elapsed time between an input time and an output time.

13. The electronic control system of claim 12, wherein the input time comprises a time when the kinematic data, surface EMG recordings, or combination thereof is received by the controller.

14. The electronic control system of claim 13, wherein the output time comprises a time by which the one or more control settings of the control device must be modified for the subject to achieve a smoother gait as compared to a gait prior to the modification.

15. The electronic control system of claim 14, wherein the smoother gait comprises the subject adapting to movement in accordance with the R terrain scenario or the T terrain scenario.

16. The electronic control system of claim 1, wherein the controller is configured to perform a binary classification of the EMG recordings and kinematic data as corresponding to the rigid (R) terrain scenario or the transition (T) terrain scenario, and dynamically modify, in real-time, the one or more control settings of the control device in response to the binary classification of the EMG recordings and kinematic data.

17. The electronic control system of claim 1, wherein the controller is configured to perform a multiclass classification of the EMG recordings and kinematic data.

18. The electronic control system of claim 17, wherein the multiclass classification includes the transition (T) terrain scenario having a plurality of levels or degrees of compliance, and the controller is configured to dynamically modify, in real-time, the one or more control settings of the control device in response to the multiclass classification of the EMG recordings and kinematic data.

19. The electronic control system of claim 1, wherein the lower limb prosthesis 9 is selected from the group consisting of: an artificial limb, an orthotic device, and an exoskeleton.

20. A powered locomotion assistance system comprising:
the electronic control system of claim 1;
the plurality of sensors attached to the subject;
the lower limb prosthesis attached to at least one connection point corresponding to a missing or compromised limb of the subject.

21. A method for providing locomotion assistance to a subject, the method comprising:
a) providing the powered locomotion assistance system of claim 20;
b) attaching the at least one powered lower limb prosthesis and the plurality of the sensors to the subject;
c) processing the kinematic data related to motion of the subject;
d) processing the EMG recordings of one or more of the subject's muscles during the at least one gait cycle;
e) performing the classification of the kinematic data and EMG recordings;
f) predicting the subject's intent to move or prepare for movement based on the classification of the kinematic data and EMG recordings; and
dynamically modifying, in real-time, the one or more control settings of the control device in response to the classification of the kinematic data and EMG recordings.
